(19) United States
(12) Patent Application Publication
(10) Pub. No.: US 2009/0304800 A1
(43) Pub. Date: Dec. 10, 2009

(54) Dry Coating using Twin-Screw Kneader

(75) Inventors: Shinji Fujimoto, Osaka (JP); Toshinobu Uemura, Osaka (JP); Kantaro Kaneko, Osaka (JP); Yoshinobu Fukumori, Hyogo (JP)

Correspondence Address: KRATZ, QUINTOS & HANSON, LLP, 1420 K Street, N.W., Suite 400, WASHINGTON, DC, 20005, US

(73) Assignee: KURIMOTO, LTD., OSAKA-SHI (JP)

(21) Appl. No.: 12/083,628

(22) PCT Filed: Oct. 12, 2006
(86) PCT No.: PCT/JP2006/320372

§ 371 Date: Jun. 23, 2009

(30) Foreign Application Priority Data
Oct. 17, 2005 (JP) 2005-301953

(51) Int. Cl. A61K 9/14; B05D 3/00

(52) U.S. Cl. 424/490; 427/2.21

(57) ABSTRACT

An object of the present invention is to provide a dry coating process that can produce a dry-coated preparation in a large amount. A large amount of dry-coated preparation can be produced more efficiently, than by prior-art processes, by a process in which a material containing core particles and a dry binder (lauric acid, myristic acid, or the like) is kneaded in a twin-screw kneader to produce dry binder particles in which the surfaces of the core particles are dry-coated with the dry binder. Further, a dry coating particle production process in which a material containing core particles, a dry binder, and a coating powder is kneaded in a twin-screw kneader can also produce a large amount of dry-coated preparation more efficiently than prior-art processes.

### TECHNICAL FIELD

The present invention relates to a process for producing a dry-coated preparation, the process being capable of continuous production.

### BACKGROUND ART

Coated preparations are used which are provided with coatings to impart various functionalities, such as gastric solubility, enteric properties, slow-release properties, etc. to preparations. The processes for applying coating agents to preparations are classified into wet processes and dry processes, among which wet processes are generally employed.

In a typical wet process, a liquid in which a coating agent is dissolved or suspended is sprayed on a preparation, and then the liquid is evaporated. However, when the solvent for the coating agent is water, there are problems in that the evaporation after spraying requires a large amount of energy, and that the use of components that are deteriorated by water is restricted since such components deteriorate if contained in the core. When an organic solvent is used as the solvent for the coating agent, there is a problem in that the organic solvent remains unless it is completely removed.

In contrast, dry processes are free from these problems since such processes do not use a solvent. However, it is difficult to apply a coating agent to a preparation without using a solvent; therefore, the use of additives such as dry binders has been attempted, but the coating efficiency is still low. Non-Patent Document 1 discloses an example in which Celphere particles are coated for fixation of a water-soluble model drug using lauric acid, and further coated with a coating powder obtained by freeze-drying an aqueous ethylcellulose suspension using a high speed elliptical-rotor type powder mixer. This process is a batch process, and therefore, for large-scale production, it is necessary to design a large-size apparatus. However, since the clearance between the high-speed rotor and rotating vessel needs to be about 1 mm, it is difficult to design a large-size apparatus. No large-scale production apparatus has so far been developed, resulting in low production efficiency for preparations. Accordingly, a more efficient process for producing a dry-coated preparation is desired.

Non-Patent Document 1: Preparation of Controlled Release Microcapsules by a High-Speed Elliptical-Rotor-Type Mixer (Book of Abstracts), Proceedings of the World Congress on Particle Technology 3, No. 121, Brighton, UK, Jul. 7-9, 1988, sponsored by the British Institute of Chemical Engineers

### DISCLOSURE OF THE INVENTION

### Problems to be Solved by the Invention

An object of the present invention is to provide a process that is capable of providing suitable coatings and producing a dry-coated preparation in a large amount.

### Means for Solving the Problems

The present inventors conducted extensive research on a large-scale production process for a dry-coated preparation, and as a result, found that a dry-coated preparation can be produced in a large amount by carrying out dry coating in a twin-screw kneader using a dry binder, thereby accomplishing the present invention.

The present invention provides the following production process, dry binder particles, and dry-coated particles.

Item 1. A process for producing dry binder particles, the process comprising kneading a material comprising core particles and a dry binder in a twin-screw kneader.

Item 2. A process according to Item 1, wherein the dry binder is obtained by micronization in a fluidized-bed jet mill.

Item 3. Dry binder particles obtained by a process according to Item 1 or 2.

Item 4. A process for producing dry-coated particles, the process comprising kneading a material comprising particles according to Item 3 and a coating powder in a twin-screw kneader.

Item 5. A process according to Item 4, wherein the coating powder is obtained by micronization in a fluidized-bed jet mill.

Item 6. Dry-coated particles obtained by a process according to Item 4 or 5.

Item 7. Dry-coated particles according to Item 6, which are a pharmaceutical preparation.

Item 8. Dry-coated particles according to Item 7, wherein the coating powder is a drug.

The present invention also provides the following production process.

Item 9. A process for producing dry-coated particles, the process comprising kneading a material comprising core particles, a dry binder, and a coating powder in a twin-screw kneader.

The dry binder particles of the present invention are obtained by dry-coating core particles with a dry binder in a twin-screw kneader. Since the particles of the present invention have coatings of a dry binder on their surfaces, dry coating with a coating powder, which has heretofore been difficult, can be very easily performed. For example, particles that are dry-coated with a coating powder can be easily produced in a large amount by supplying the dry binder particles and the coating powder to a twin-screw kneader and kneading them. The dry-coated particles can also be produced by supplying core particles, a dry binder, and a coating powder to a twin-screw kneader at the same time and kneading them.

The dry-coated particles may be further coated with a coating powder in a twin-screw kneader or other dry coating apparatus. The resulting dry-coated particles may be further coated with a dry binder in a twin-screw kneader, and the resulting dry binder-coated particles may be further coated with a coating powder in a twin-screw kneader.

The dry-coated particles is further coated as required, and can be used in fields that employ coated particles, such as the fields of pharmaceuticals, foods, agricultural chemicals, feedstuff, chemistry, etc. The coating powder can be suitably selected according to the intended use of the particles. For example, for pharmaceutical use, a drug can be used as the coating powder.

The production process of the present invention is described with reference to FIG. 1. FIG. 1 is a side sectional view of a typical continuous twin-screw kneader (KRC-S1, produced by Kurimoto, Ltd.). FIG. 2 is a front sectional view of the kneader. Since the kneader is structured so that paddles and screw member provided on two shafts are engaged, it has good mixing properties, good applicability to adhesive powders, and good temperature controllability. Table 1 shows the main specifications of this kneader.

TABLE 1

| | |
|---|---|
| Paddle Dimensions | 25 mm (diameter) × 255 mm (L/D = 10.2) |
| Effective Volume | 120 cm³ |
| Material | Casing: SUS329 |
| | Paddle: SUS316 |
| Paddle Rotation Number | 50 to 300 rpm |
| Temperature Control | Possible |
| Dimensions | 1650 mm (length) × 700 mm (width) × |
| | 1800 mm (height) |
| Approximate Weight | 500 kg |

The left end of the kneader is a preparation outlet 7. One shaft of the kneader is provided with a discharge screw member 1, a plurality of spindle-shaped paddles 2, and a feed screw member 5, disposed in that order from the side closest to the outlet 7. FIG. 2 shows that the paddles 2 are spindle-shaped. The kneader has another shaft provided with a discharge screw member, paddles, and a feed screw member, although it is not shown in FIG. 1. The two shafts are disposed so that the discharge screw member, paddles, and feed screw member provided on one shaft are engaged with the discharge screw member, paddles, and feed screw member on the other shaft, respectively. FIG. 2 shows that the paddles are engaged with each other. The casing 4 has, at the portion surrounding the shafts, a jacket structure provided with a temperature control space 3, through which warm water, cold water, refrigerant, or the like is supplied as required, so as to maintain the interior of the casing 4 at a temperature necessary for coating. Further, a heater can be provided on the outer face of the casing. Provision of a heater enables temperature control up to a maximum of 300° C.

When a material is supplied from a material feed port 6 disposed above the feed screw members 5, the material, while being mixed, is conveyed by the feed screw members 5 toward the outlet 7. The material conveyed is kneaded by the rotations of the paddles 2, and the particle surfaces are coated with a coating component (dry binder, coating powder, or the like). The preparation that has been coated is discharged by the discharge screw members 1 from the outlet 7 so as to produce a coated preparation. The residence time in the kneader varies depending on the paddle rotation number, but is usually about several tens of seconds to about several minutes.

Usable twin-screw kneaders include general twin-screw kneaders, such as the twin-screw kneader disclosed in Japanese Patent No. 3590542 and other documents. A continuous twin-screw kneader is advantageous for large-scale production. It is especially preferable to use a kneader in which screw members and paddles that are engaged are disposed on two shafts in the order of screw members (feed screw members), paddles, and screw members (discharge screw members), as viewed from the material feed port side to the outlet side.

The dry binder particle production process of the present invention comprises kneading a material comprising core particles and a dry binder in a twin-screw kneader. The core particle content in the material is not limited, and is usually 70 to 95 wt. %, and preferably 75 to 95 wt. %. The dry binder content in the material is also not limited, and is usually 5 to 30 wt. %, and preferably 5 to 25 wt. %. This production process can produce particles continuously by supplying the material continuously.

The temperature during kneading is not limited, and is usually close to but not exceeding the melting point of the dry binder. The temperature during kneading is preferably 0.5 to 10° C. lower, and more preferably 0.5 to 7° C. lower, than the melting point. The paddle rotation number per minute is not limited, and is usually 50 to 300, and preferably 100 to 300. The supply rate of the material is not limited and varies depending on the scale of the kneader. When using KRC-S1, the supply rate is usually 5 g to 50 g per minute, and preferably 6 g to 40 g per minute.

The core particles may be an active ingredient (e.g., a drug in a pharmaceutical preparation), a mixture of a carrier and a drug, carrier particles that are surface-coated with a drug, or a carrier containing no drug. The core particles are not limited as long as they are not disintegrated during the process. The core particles have a mean particle diameter of preferably 30 to 1000 μm, and more preferably 50 to 500 μm, although these ranges are not limiting. Examples of usable core particles include pills, granules, powders, single crystals of drugs, aggregates of drug powders, lactose particles, hydroxyapatites, calcium carbonate particles, products marketed as coating core particles in the field of pharmaceutical preparations, such as crystalline cellulose granules (Celphere, produced by Asahi Kasei Corp.), sucrose spherical granules and mannitol spherical granules (both available under the tradename “NonPareil”, produced by Freund Industrial Co., Ltd.), etc. The core particles may be a controlled-release preparation, such as a rapid-release preparation, sustained-release preparation (slow-release preparation), or the like. The core particles may contain conventional additives and can be produced by a known method. Examples of such additives include excipients, disintegrators, binders, lubricants, coloring agents, pH control agents, surfactants, release-retarding agents, stabilizers, acidulants, flavors, fluidizing agents, etc. These additives are used in conventional amounts in the field of pharmaceutical preparations.

Examples of drugs that serve as active ingredients of pharmaceutical preparations include central nervous system drugs, such as aspirin, indomethacin, ibuprofen, naproxen, diclofenac sodium, meclofenoxate hydrochloride, chlorpromazine, tolmetin sodium, milnacipran hydrochloride, phenobarbital, etc.; peripheral nervous system drugs, such as etomidoline, tolperisone hydrochloride, pipethanate ethobromide, methylbenactyzium bromide, flopropion, etc.; hemostatics, such as carbazochrome sodium sulfonate, protamine sulfate, etc.; circulatory system drugs, such as aminophylline, etilefrine hydrochloride, diltiazem hydrochloride, digitoxin, captopril, etc.; respiratory system drugs, such as ephedrine hydrochloride, clorprenaline hydrochloride, oxeladin citrate, cloperastine, sodium cromoglycate, etc.; digestive system drugs, such as berberine chloride, loperamide hydrochloride, cimetidine, ranitidine hydrochloride, famotidine, etc.; coronary vasodilators, such as nifedipine, nicardipine, verapamil, etc.; vitamins, such as ascorbic acid, thiamin hydrochloride, calcium pantothenate, riboflavin butyrate, etc.; metabolic preparations, such as camostat mesilate, mizoribine, lysozyme chloride, etc.; antiallergic agents, such as cyproheptadine hydrochloride, diphenhydramine hydrochloride, alimemazine tartrate, suplatast tosilate, diphenhydramine maleate, etc.; chemotherapeutic agents, such as acyclovir, enoxacin, ofloxacin, pipemidic acid trihydrate, levofloxacin, etc.; and antibiotics, such as erythromycin, cefcapene pivoxil hydrochloride, cefteram pivoxil, cefpodoxime proxetil, cefaclor, cephalexin, clarithromycin, rokitamycin, etc.

Examples of excipients include starches, such as corn starch, potato starch, wheat starch, rice starch, partially gelatinized starch, gelatinized starch, porous starch, etc.; sugars and sugar alcohols, such as lactose, fructose, glucose, D-mannitol, sorbitol, etc.; anhydrous calcium phosphate; crystalline cellulose; precipitated calcium carbonate; calcium silicate; etc.

Examples of disintegrators include carboxymethylcellulose, carboxymethylcellulose calcium, sodium carboxymethyl starch, croscarmellose sodium, crospovidone, low-substituted hydroxypropylcellulose, hydroxypropyl starch, etc. The amount of disintegrator to be used is preferably 0.5 to 25 parts by weight, and more preferably 1 to 15 parts by weight, per 0.100 parts by weight of solid preparation.

Examples of binders include crystalline cellulose, hydroxypropylcellulose, hydroxypropylmethylcellulose, polyvinyl pyrrolidone, powdered acacia, etc. The amount of binder to be used is preferably 0.1 to 50 parts by weight, and more preferably 0.5 to 40 parts by weight, per 100 parts by weight of solid preparation.

Preferable examples of lubricants include magnesium stearate, calcium stearate, talc, sucrose fatty acid esters, sodium stearyl fumarate, etc.

Examples of coloring agents include food colors, such as Food Yellow No. 5, Food Red No. 2, Food Blue No. 2, etc.; food lake colors; iron sesquioxide; etc. Examples of pH control agents include citric acid salts, phosphoric acid salts, carbonic acid salts, tartaric acid salts, fumaric acid salts, acetic acid salts, amino acid salts, etc.

Examples of surfactants include sodium lauryl sulfate, polysorbate 80, polyoxyethylene (160) polyoxypropylene (30) glycol, etc.

Examples of stabilizers include tocopherol, tetrasodium edetate, nicotinamide, cyclodextrins, etc.

Examples of acidulants include ascorbic acid, citric acid, tartaric acid, malic acid, etc.

Examples of flavors include menthol, peppermint oil, lemon oil, vanillin, etc.

Examples of fluidizing agents include light anhydrous silicic acid, hydrous silicon dioxide, etc.

The dry binder facilitates dry coating in a twin-screw kneader and is very important in the production process of the present invention. This is because the dry binder exhibits its binding action when heated during the production, thereby facilitating dry coating. The dry binder has a mean particle diameter of preferably 1 to 100 μm, more preferably 1 to 50 μm, and still more preferably 1 to 20 μm. It is preferable to use, as the dry binder, at least one waxy substance selected from the group consisting of organic fatty acids (lauric acid, palmitic acid, myristic acid, stearic acid, etc.), ester derivatives of organic fatty acids, higher alcohols (cetyl alcohol, stearyl alcohol, etc.), glycerol fatty acid esters (glyceryl monostearate and the like), polyethylene glycols (Macrogol 6000 and the like), natural waxes (carnauba wax, rice wax, etc.), and the like. Among these, lauric acid, myristic acid, Macrogol 6000, etc. are particularly preferable, because they have a melting point of about 44 to about 60° C. and thus make it easy to control the temperature during the production, and because they have excellent binder properties.

The mean particle diameter of the particles obtained by the dry binder particle production process of the present invention is not limited, and is usually 40 to 1050 μm, and preferably 60 to 550 μm.

One embodiment of the dry-coated particle production process of the present invention comprises kneading a material comprising the dry binder particles described above and a coating powder in a twin-screw kneader. The dry binder particle content in the material is not limited, and is usually 40 to 98 wt. %, and preferably 50 to 95 wt. %. The coating powder content in the material is also not limited, and is usually 2 to 60 wt. %, and preferably 5 to 50 wt. %. According to this production process, the particles can be produced continuously by supplying the material continuously.

The temperature during kneading is not limited, and is usually close to the melting point of the dry binder. The temperature during kneading is preferably 0.5 to 15° C. lower, and more preferably 1 to 10° C. lower, than the melting point. The paddle rotation number per minute is not limited, and is 50 to 300, and preferably 100 to 300. The supply rate of the material is not limited and may vary depending on the scale of the kneader. When using KRC-S1, the supply rate is usually 5 g to 50 g per minute, and preferably 6 to 40 g per minute.

Another embodiment of the dry-coated particle production process of the present invention comprises kneading a material comprising core particles, a dry binder, and a coating powder in a twin-screw kneader. The core particle content in the material is not limited, and is usually 20 to 95 wt. %, and preferably 30 to 90 wt. %. The dry binder content in the material is not limited, and is usually 5 to 40 wt. %, and preferably 10 to 30 wt. %. The coating powder content in the material is not limited, and is usually 2 to 60 wt. %, and preferably 5 to 50 wt. %. According to this production process, the particles can be produced continuously by supplying the material continuously. The kneading conditions (temperature, paddle rotation number, etc.) are the same as those for the other embodiment of the dry-coated particle production process.

The coating powder has a mean particle diameter of preferably 0.1 to 20 μm, and more preferably 0.1 to 10 μm, but these ranges are not limiting. Examples of the coating powder include coating polymers used in the pharmaceutical field to impart slow-release properties, and also include active ingredients, such as drugs. The coating powder is not limited as long as it can provide coatings. Dry coating with a coating powder is not limited to once, and can be carried out two or more times if necessary. Further, if necessary, after dry coating with a coating powder, the resulting coated particles may be dry-coated with a dry binder. That is, the numbers of dry coating with a dry binder or coating powder can be determined as needed.

Usable examples of coating powders include coating polymers used in the fields of pharmaceutical preparations and the like, the above-mentioned drugs, etc., and also include the above-mentioned additives. Whether the active ingredients, additives, etc. are contained in the core particles or they are contained in the coating powder can be selected according to the properties, intended use, and the like of the particles to be produced.

Examples of usable coating polymers include cellulose-based polymers, acrylic-based polymers, biodegradable polymers, polyvinyl-based polymers, etc. These can be used singly or in combination. Examples of usable bases for the coating powder include cellulose-based polymers, acrylic-based polymers, biodegradable polymers, etc. These can be used singly or in combination.

Examples of cellulose-based polymers include ethylcellulose powders (e.g., STD premium FP, produced by Dow Chemical Co.), hydroxypropylmethylcellulose phthalate, hydroxypropylmethylcellulose acetate succinate, polyvinyl acetal diethylaminoacetate, carboxymethylethylcellulose, cellulose acetate phthalate, etc. Among these, ethylcellulose and hydroxypropylmethylcellulose phthalate are preferable.

Examples of acrylic-based polymers include Eudragit polymers, such as aminoalkyl methacrylate copolymers E (E100 and EPO), methacrylic acid-methyl methacrylate copolymers L (L100 and L100-55), methacrylic acid-methyl methacrylate copolymers S(S-100), aminoalkyl methacrylate copolymers RL (RL100 and RLPO), aminoalkyl methacrylate copolymers RS (RS100 and RSPO), etc. Among these, Eudragit EPO, L100, L100-55, S-100, RLPO, and RSPO are preferable.

Examples of usable biodegradable polymers include homopolymers and copolymers of L-lactic acid, D-lactic acid, DL-lactic acid, glycolic acid, ε-caprolactone, N-methylpyrrolidone, etc.; mixtures of these polymers; polycaprolactam; chitin, chitosan; etc.

Examples of polyvinyl-based polymers include polyvinyl acetal diethylaminoacetate (e.g., AEA, produced by Sankyo Co., Ltd.), PVA copolymer (produced by Nisshin Kasei Co., Ltd.), etc. An aggregation inhibitor, such as talc, sodium chloride, sodium citrate, light anhydrous silicic acid, or the like, may be added to the coating powder to prevent adhesion caused by static electricity during dry coating. The amount of aggregation inhibitor to be used is usually 5 to 40 wt. %, and preferably 10 to 30 wt. %, relative to the weight of the coating powder.

In the present invention, the coating powder and the dry binder are preferably obtained by micronization using a fluidized-bed jet mill. For example, a fluidized-bed jet mill with a built-in classification rotor (Pocket Jet, produced by Kurimoto, Ltd.) can be used. When using a fluidized-bed jet mill with a built-in classification rotor as a fluidized-bed jet mill, the coating powder and dry binder are obtained as micronized products in which the amount of course particles has been reduced classification ro. Since such a micronized product has a narrow particle size distribution, the micronized product provides more uniform coatings when it forms an ordered mixture on the core particles. This suppresses adhesion and aggregation of particles and makes it possible to efficiently produce single-core particles. Thus, the use of a fluidized-bed jet mill with a built-in classification rotor is preferable.

The mean particle diameter of the preparation obtained by the dry-coated preparation production process of the present invention is not limited, but is preferably 50 to 1150 μm, and more preferably 70 to 530 μm. The dry-coated preparation of the present invention may be dry-coated with a known coating substance using a known dry coating apparatus other than the twin-screw kneader described above. Examples of such dry coating apparatus include vortex mixers, vibration mills, V-shaped mixers, centrifugal rotation mixers (Mechanomill, produced by Okada Seiko Co., Ltd.), etc.

### EFFECTS OF THE INVENTION

According to the present invention, a dry-coated preparation can be easily produced in a large amount.

### BEST MODE FOR CARRYING OUT THE INVENTION

The following examples are given to illustrate the present invention in further detail, and are not intended to limit the scope of the invention.

### EXAMPLES

### Example 1

### Dry Coating with Dry Binder

The material described below was supplied to a continuous twin-screw kneader (KRC-S1, produced by Kurimoto, Ltd.) to produce dry binder particles. The specifications of the kneader are as shown in Table 1 except for the temperature and the paddle rotation number. Before production, the core particles alone were supplied to the kneader, and it was confirmed that no destruction or breakage of the core particles was observed.

### Material

Core particles: Crystalline cellulose spherical granules (hereinafter sometimes referred to as CP) (Celphere CP-102, produced by Asahi Kasei Corp.), which were used without classification.

The particle size distribution of the classified fractions of CP-102 (Ro-Tap Shaker) is as follows. 74 to 106 μm: 0.3%; 106 to 150 μm: 37.8%; 150 to 177 μm: 36.5%; 177 to 210 μm: 24.1%; 210 to 250 μm: 1.3%.

Dry binder: Lauric acid (produced by Wako Pure Chemical Industries, Ltd.; melting point: 44° C.; hereinafter sometimes referred to as LA); fine particles with a mean particle diameter (measured by a laser scattering type particle size measuring apparatus, LDSA-2400A, produced by Tohnichi Computer Applications Co., Ltd.) of 5.5 μm, obtained by operating a fluidized-bed jet mill with a built-in classification rotor (Pocket Jet, produced by Kurimoto, Ltd.) at a classification rotor speed of 8000 rpm.

500 g of CP and 60 g of LA were mixed in a plastic bag for 5 minutes, and supplied to the twin-screw kneader through a gravimetric feeder (produced by KUMA) at a supply rate of 39 g/min. The twin-screw kneader was set to a temperature of 43° C. and a paddle rotation number of 200 rpm, and continuously operated for about 15 minutes.

CP that were dry-coated with LA (hereinafter sometimes referred to as CP/LA) was thus obtained. The LA coating ratio of CP/LA was determined as follows. LA that did not form a coating was first removed using an air jet sieve (produced by ALPINE; 10 inches H₂O, 3 minutes) with a 63 μm mesh screen, and the amount of LA that did not form a coating was calculated from the weight difference. To calculate the coating ratio, the difference between the amount of supplied LA and the amount of LA that did not form a coating was calculated as the amount of LA that formed coatings. As a result, the LA coating ratio was 94.1%.

Microscopic observation of the obtained particles showed no aggregation of LA alone, and revealed that CP was coated with LA. Further, the particles were sieved using a Ro-Tap Shaker for 10 minutes to obtain a fraction with a particle diameter of 250 μm or more as aggregated particles. The aggregation ratio was found to be 2.4%. That is, 533 g of particles with a LA coating ratio of 94.1% and an aggregation ratio of 2.4% was obtained in an operation time of about 15 minutes.

### Example 2

### Dry Coating with Dry Binder

Setting the twin-screw kneader to a temperature of 42.5° C., a paddle rotation number of 200 rpm, and a supply rate of 21 g/min, CP/LA was produced by following the procedure of Example 1. The LA coating ratio was 91.9%, and the aggregation ratio was 0.7%.

### Example 3

### Dry Coating with Dry Binder

Using 200 g of CP and 30 g of LA as the material (the LA content in the material: 13 wt. %) and setting the twin-screw kneader to a temperature of 42.5° C., a paddle rotation number of 200 rpm, and a supply rate of 14 g/min, CP/LA was produced by following the procedure of Example 1. The LA coating ratio was 90.3% and the aggregation ratio was 0%.

### Example 4

### Dry Coating with Dry Binder

Using 500 g of the CP described below as core particles and 60 g of LA, and setting the twin-screw kneader to a temperature of 42.5° C., a paddle rotation number of 200 rpm, and a supply rate of 43 g/min, CP/LA was produced by following the procedure of Example 1. The yield was 549 g, the LA coating ratio was 95.3%, and the aggregation ratio (calculated from the ratio of the classified fraction having a particle diameter of 355 μm or more) was 0.3%.

Core particles: crystalline cellulose spherical granules (Celphere CP-203, produced by Asahi Kasei Corp.), which were used without classification.

The particle size distribution of the classified fractions of CP-203 (Ro-Tap Shaker) was as follows. 177 to 210 μm: 7.2%; 210 to 250 μm: 68.9%; 250 to 297 μm: 23.9%.

### Example 5

### Dry Coating with Dry Binder

Using 100 g of the CP-203 described in Example 4 as core particles and 12 g of myristic acid described below (hereinafter sometimes referred to as MA) as a dry binder, and setting the twin-screw kneader to a temperature of 52.5° C., a paddle rotation number of 200 rpm, and a supply rate of 19.5 g/min, CP/MA was produced by following the procedure of Example 1. The yield was 114 g, the MA coating ratio was 96.7%, and the aggregation ratio was 0.1%.

Dry binder: myristic acid (produced by Wako Pure Chemical Industries, Ltd.; melting point: 58° C.); fine particles having a mean particle diameter of 9.0 μm obtained by operating a fluidized-bed jet mill with a built-in classification rotor (Pocket Jet, produced by Kurimoto, Ltd.) at a classification rotor speed of 15000 rpm.

### Example 6

### Dry Coating with Dry Binder

Using 100 g of the CP-203 described in Example 4 as core particles and 12 g of the polyethylene glycol 6000 described below (hereinafter sometimes referred to as PEG6000) as a dry binder, and setting the twin-screw kneader to a temperature of 51.0° C., a paddle rotation number of 300 rpm, and a supply rate of 17.4 g/min, CP/PEG6000 was produced by following the procedure of Example 1. The yield was 105 g, the PEG6000 coating ratio was 93.0%, and the aggregation ratio was 0.6%.

Dry binder: PEG6000 (produced by Wako Pure Chemical Industries, Ltd.; melting point: 56 to 61° C.); fine particles having a mean particle diameter of 6.7 μm obtained by operating a fluidized-bed jet mill with a built-in classification rotor (Pocket Jet, produced by Kurimoto, Ltd.) at a classification rotor speed of 6000 rpm.

### Example 7

### Dry Coating with Coating Powder

The CP/LA obtained in Example 1 was sieved using an air jet sieve fitted with a 63 μm mesh screen, and the particles having a diameter of 250 μm or more were removed by classification. The remaining particles were mixed with the drug described below, i.e., carbazochrome sodium sulfonate (hereinafter sometimes referred to as CCSS). The amount of CCSS added corresponds to 10.7 wt. % of CP/LA. The mixture was supplied to the twin-screw kneader in the same manner as in Example 1, to produce CP/LA coated with CCSS (hereinafter sometimes referred to as CP/LA/CCSS). The kneading temperature was 42.8° C., the supply rate was 16 g/min, and the paddle rotation number was 200 rpm. The CCSS coating ratio was 89.7%, and the aggregation ratio was 1.7%.

Coating powder: Fine particles having a mean particle diameter of 4.0 μm obtained by processing carbazochrome sodium sulfonate (produced by Sanwa Chemical Co., Ltd.), which is a water-soluble drug, using a fluidized-bed jet mill with a built-in classification rotor (Pocket Jet, produced by Kurimoto, Ltd.) at a classification rotor speed of 15000 rpm.

### Example 8

### Dry Coating with Coating Powder

The CP/LA obtained in Example 3 was sieved using an air jet sieve fitted with a 63 μm mesh screen, and particles with a diameter of 355 μm or more were removed by classification. The remaining particles were mixed with CCSS. The amount of CCSS added corresponds to 10.7 wt. % of the CP/LA. The mixture was supplied to the twin-screw kneader in the same manner as in Example 1, to produce CP/LA/CCSS. The kneading temperature was 42.5° C., the supply rate was 16.5 g/min, and the paddle rotation number was 250 rpm. The CCSS coating ratio was 91.2%, and the aggregation ratio was 5.1%.

### Example 9

### Dry Coating with Coating Powder

The CP/LA obtained in Example 4 was sieved using an air jet sieve fitted with a 63 μm mesh screen, and particles with a diameter of 355 μm or more were removed by classification. The remaining particles were mixed with CCSS. The amount of CCSS added corresponds to 10.7 wt. % of the CP/LA. The mixture was supplied to the twin-screw kneader in the same manner as in Example 1, to produce CP/LA/CCSS. The kneading temperature was 42.5° C., the supply rate was 16 g/min, and the paddle rotation number was 275 rpm. The CCSS coating ratio was 89.3%, and the aggregation ratio was 0.4%.

### Example 10

### Simultaneous Dry Coating with Dry Binder and Coating Powder

The CP and LA used in Example 1 and the CCSS used in Example 7 were mixed at a weight ratio of 87:8:5, and supplied to the twin-screw kneader in the same manner as in Example 1, to produce CP/LA/CCSS. The kneading temperature was 42.7° C., the supply rate was 29 g/min, and the paddle rotation number was 200 rpm. The ratio of coating with LA and CCSS as coating powders was 91.8%, and the aggregation ratio was 6.0%.

### Comparative Example 1

### Dry Coating by Prior Art Process

The process described in Non-Patent Document 1 was performed. CP having a particle diameter of 150 to 170 μm was used as core particles. LA that had been micronized using a hammer mill (produced by Fuji Paudal Co., Ltd.) and classified into a particle diameter of 63 μm or less was used. CCSS that had been micronized using a planetary ball mill (Pulverisette-7, produced by Fritsch, Germany) and classified into a particle diameter of 63 μm or less was used. The mean particle diameters of LA and CCSS were 21.3 μm and 5.4 μm, respectively (measured using a laser scattering type particle size measuring apparatus, LDSA-2400A, produced by Tohnichi Computer Applications Co., Ltd.).

CP was coated with LA using a high-speed elliptical agitation mixer (Theta Composer, produced by Tokuju Corp.). 25 g of CP and 3 g of LA were supplied to the material feed portion between the rotor and the vessel, and the rotation speed of the vessel was set to 20 rpm. The rotor was operated at speeds of 500 rpm (2 minutes), 1000 rpm (3 minutes), 2000 rpm (5 minutes), and 3000 rpm (5 minutes) in that order, to increase the shear force. Subsequently, coating was performed for 90 minutes at an increased vessel rotation speed of 30 rpm, to produce CP/LA. As a result, the yield was 27.3 g and the LA coating ratio was 85%. The coating time required to reach this stage was 105 minutes.

The resulting CP/LA was then coated with CCSS. 25 g of CP/LA and 3 g of CCSS were supplied to the above mixer set to a vessel rotation number of 20 rpm. The rotor was operated at speeds of 500 rpm (2 minutes), 1000 rpm (3 minutes), 1500 rpm (5 minutes), and 2000 rpm (135 minutes) in that order, to obtain CP/LA/CCSS. The yield of CP/LA/CCSS was 27.4 g, and the coating ratio was 89.7%. The CCSS coating time was 145 minutes.

### Test Example 1

The following test was performed to examine the pharmaceutical usefulness of CP/LA obtained using the continuous twin-screw kneader.

Non-Patent Document 1 discloses an example using LA and CCSS and using ethylcellulose (hereinafter sometimes referred to EC) as a fine particulate polymer.

A preparation was produced by dry-coating the CP/LA/CCSS obtained in Example 8 with EC. EC (Ethocel 7FP, produced by Dow Chemical Co.) that had been micronized using a jet mill (classification rotor: 15000 rpm) into fine particles having a mean particle diameter of 2.5 μm was used.

The CP/LA/CCSS obtained in Example 8 was sieved using an air jet sieve fitted with a 63 μm mesh screen to remove particles that did not form a coating, and then aggregated particles having a diameter of 250 μm or more were removed by sieving. 20 g of the resulting CP/LA/CCSS and 2.5 g (corresponding to 11.1 wt. %) of EC were placed in a 50-ml standard No. 7 bottle, mixed in a vortex mixer (Automatic Lab-mixer HM-10, produced by Iuchi Co.) for 1 minute to obtain coated particles (hereinafter sometimes referred to as CP/LA/CCSS/EC). CP/LA/CCSS/EC at this stage is referred to as P1. Microscopic observation revealed that EC efficiently formed coatings.

2.5 g of the obtained CP/LA/CCSS/EC was sampled and supplied together with 1.28 g (corresponding to 6.0%) of EC, and the same operation was performed to increase the amount of EC coating. CP/LA/CCSS/EC obtained at this stage is referred to as P2. Further, 3 g of CP/LA/CCSS/EC was sampled and coated with 1.28 g (corresponding to 6.5 wt. %) of EC by performing the same operation. CP/LA/CCSS/EC obtained at this stage is referred to as P3.

Samples of P1 to P3 were passed through a 355 μm sieve to remove aggregated particles of only ethylcellulose, which were observed in a small amount. The CCSS content was measured (363 nm) to determine the coating content. The ethylcellulose coating amounts of the three samples were 10.7 wt. %, 16.4 wt. %, and 21.9 wt. %, respectively.

Each of P1 to P3 was separately mixed with 1 wt. % light anhydrous silicic acid (Aerosil #200, produced by Japan Aerosil Co.), and cured by heating at 40° C. for 3 hours and then at 60° C. for 3 hours. That is, the preparations were placed in sample bottles, and Aerosil was added and mixed by shaking by hand. The apparatus used for curing was a mini-jet oven. The samples were heated to 40° C. in the mini-jet oven, mixed by shaking every about 5 minutes 5 times to prevent adhesion of the preparation particles, and then allowed to stand. The samples were subsequently cured at 60° C. for 3 hours in the same manner. After completion of the heating, the samples were cooled while being shaken by hand to prevent adhesion. Thereafter, the samples were sieved using an air jet sieve (Alpine 200LS) equipped with a 63 μm mesh screen to remove light anhydrous silicic acid. The obtained preparations were subjected to the dissolution test of the second method (paddle method, 100 rpm) specified in the Japanese Pharmacopoeia. 900 mL of distilled water was used as the dissolution test liquid. The concentration of CCSS, a medicinal component, was determined from the absorbance (363 nm). FIG. 3 shows the results of the dissolution test. As the coating amount increased, the dissolution speed of CCSS decreased. This demonstrates that the dissolution speed can be controlled by changing the coating amount.

### INDUSTRIAL APPLICABILITY

The present invention can be applied in fields, that employ coated particles, such as the fields of foods, agricultural chemicals, feedstuff, chemistry, etc. For example, the present invention is applicable for drug layering process, sustained-release preparations, taste-masking preparations, etc.

### BRIEF DESCRIPTION OF THE DRAWINGS

FIG. 1 is a side sectional view of a typical continuous twin-screw kneader (KRC-S1, produced by Kurimoto, Ltd.).

FIG. 2 is a front sectional view of a typical continuous twin-screw kneader (KRC-S1, produced by Kurimoto, Ltd.), cut at the paddle position.

FIG. 3 is a graph showing the results of the dissolution test in Test Example 1. The ordinate indicates the CCSS dissolution amount, and the abscissa indicates the dissolution time.

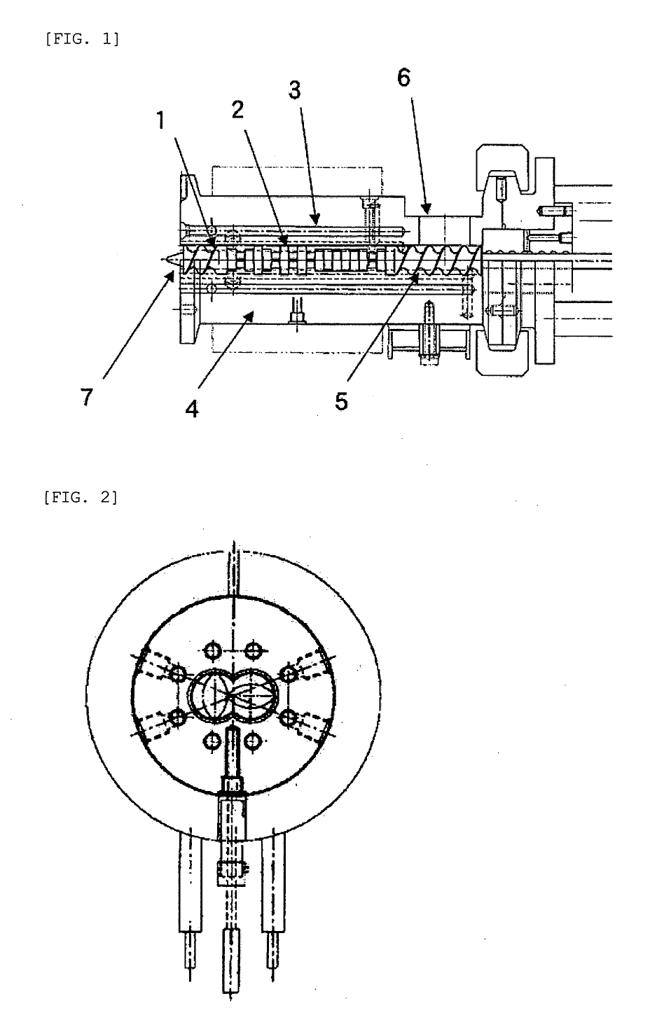

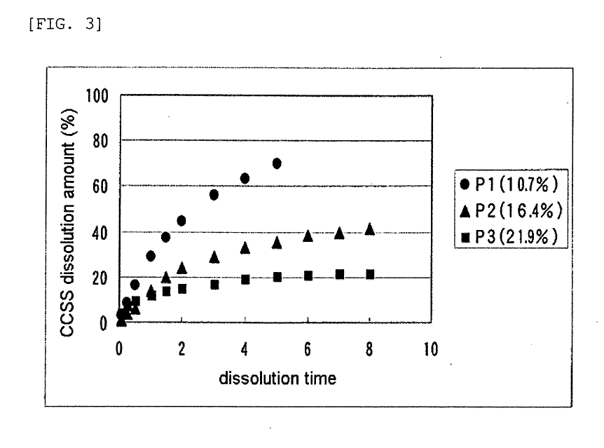

### DESCRIPTION OF THE NUMERALS

1 Discharge Screw Member
2 Paddle
3 Temperature Control Space
4 Casing
5 Feed Screw Member
6 Material Feed Port
7 Preparation Outlet

1. A process for producing a slow-release pharmaceutical preparation, the process comprising the steps of kneading a material comprising core particles and a dry binder in a twin-screw kneader to obtain dry binder particles, and kneading a material comprising the obtained dry binder particles and a coating powder in a twin-screw kneader to obtain dry-coated particles; at least either the core particles or the coating powder comprising a drug.

2. A process according to claim 1, wherein the dry binder and/or the coating powder is obtained by micronization in a fluidized-bed jet mill.

3. A process according to claim 2, wherein the fluidized-bed jet mill is a fluidized-bed jet mill with a built-in classification rotor.

4. A process according to claim 3, further comprising the step of kneading, in a twin-screw kneader, a material comprising the dry-coated particles and a coating polymer that can be used in the pharmaceutical field to impart slow-release properties, to obtain further coated dry-coated particles.

5. A slow-release pharmaceutical preparation obtained by a process according to claim 4.

6. (canceled)

7. (canceled)

8. (canceled)

9. (canceled)

10. A process according to claim 1, further comprising the step of kneading, in a twin-screw kneader, a material comprising the dry-coated particles and a coating polymer that can be used in the pharmaceutical field to impart slow-release properties, to obtain further coated dry-coated particles.

11. A slow-release pharmaceutical preparation obtained by a process according to claim 10.

12. A process according to claim 2, further comprising the step of kneading, in a twin-screw kneader, a material comprising the dry-coated particles and a coating polymer that can be used in the pharmaceutical field to impart slow-release properties, to obtain further coated dry-coated particles.

13. A slow-release pharmaceutical preparation obtained by a process according to claim 12.

14. A slow-release pharmaceutical preparation obtained by a process according to claim 1.

15. A slow-release pharmaceutical preparation obtained by a process according to claim 2.

16. A slow-release pharmaceutical preparation obtained by a process according to claim 3.
